(19) United States
(12) Patent Application Publication
(10) Pub. No.: US 2014/0012104 A1
(43) Pub. Date: Jan. 9, 2014

(54) Method for Observing, Identifying, and Detecting Blood Cells

(71) Applicant: National Taiwan University, Taipei City (TW)

(72) Inventors: Chien-Kuo CHEN, Taipei City (TW); Tzu-Ming LIU, Taipei City (TW)

(73) Assignee: National Taiwan University, Taipei City (TW)

(21) Appl. No.: 13/973,873

(22) Filed: Aug. 22, 2013

(30) Foreign Application Priority Data
Feb. 21, 2012 (TW) 102105993

(51) Int. Cl. A61B 5/1455

(52) U.S. Cl. CPC A61B 5/1455
USPC 600/322

(57) ABSTRACT

The invention provides a method for the observation, identification, and detection of blood cells, which comprises a label-free third harmonic generation (THG) tomography having a property of least injury. Submicron morphologies and granularities of blood cells can be revealed and reflected through this method. Leukocytes with different granularities can thus be identified from the intensity and distribution of third harmonic generation signals generated within cells. Furthermore, the method of the present invention is capable of performing a noninvasive sectioning microscopy image in vivo. Without cell and tissue damage, label-free third harmonic generation microscopy can real-time observe the morphology and dynamics of blood cells flowing in vessels or trafficking in tissues; Red blood cells and leukocytes have different morphology in blood flow and can thus be distinguished by in vivo third harmonic generation microscopy.

### CROSS REFERENCE TO RELATED APPLICATION

This application claims the benefits of the Taiwan Patent Application Serial Number 102105993, filed on Feb. 21, 2013, the subject matter of which is incorporated herein by reference.

### BACKGROUND OF THE INVENTION

1. Field of the Invention

The present invention relates to a method for observation, identification, and detection of microscopic images of blood cells, more particularly, to a method for observation, identification, and detection of microscopic images of fast moving blood cells in vivo.

2. Description of Related Art

Currently, microscopic images, differentiation of types, and counts of blood cells can be detected after a draw of blood. If informations of blood cells are needed to be retrieved by a noninvasive way, a skin check or measurement through an optical instrument would be necessary. The flow morphology of red blood cells in human capillary could be observed by using strong absorption contract of hemachrome through a traditional white light photomicrography, but leukocytes without hemachrome may not be observed. The leukocytes in the tissues could be observed by scattering contrast through a reflective confocal microscopy, but in the depth of 100 to 150 μm of human capillary, three dimensional resolutions and image contrasts of leukocytes deteriorate rapidly due to scatterance of epidermal layers so as to the types of blood cells cannot judge precisely.

Recently, new articles report that different types of blood cells in vivo may be observed by using a multi-color confocal microscopy, and also assert that leukocytes with granularities may be recognized from blood flow. However, the technology may only be applied to mucosa in mouth, without scattering loss from pigments, of which observing locations is very inconvenient for general routine examinations, and sensitivities of lymphocytes and leukocytes having lower granules may not still be recognized. Up to now, commonly used imaging technologies for blood cell counting are only applied by a label or a flow cytometry in vitro through physical parameters of linear optics, or these images are captured outside the human body to recognize types of leukocytes. These few optical parameters may be applied to recognize types of blood cells in vivo, but they are all performed with labels or in small animals. They will have toxicity concern in clinical use. Therefore, identifications and counts of leukocytes may not be completely achieved in vivo, especially for clinical use, by these methods.

To solve the above problems, the inventor of the present application provides a method for counting and type identification of leukocytes and red blood cells without a draw of blood. The red blood cells and leukocytes in vivo may be observed without labeling by means of a noninvasive high speed third harmonic generation microscopy. The sub-cellular details of blood cells in vivo were better resolved than previous works. The leukocytes can thus be identified from a flow of red blood cells by the microscopic images, because the tumbling and flowing morphological dynamics of disc-shaped red blood cells, lacking of nuclei, and leukocytes have obvious differences in symmetry. At the same time, granularities of leukocytes could be reflected by the intensity and distribution of third harmonic generation signals generated within cells, and then leukocytes to be high granularity leukocytes or low granularity lymphocyte could be identified.

### SUMMARY OF THE INVENTION

The object of the present invention is to provide a method for observing, identifying, and detecting blood cells with minor injury, and without staining steps or labeling by way of optical Tomography. Through the method of the present invention, a virtual optical biopsy of submicron-level to show morphologies and granularities of cells without redundant labeling in a noninvasive way can be provided through the optical contrast of third harmonic generation. Moreover, the images of the blood vessels and blood cells therein in vivo under human skin with high resolution of submicron-level, which is rarely obtained through other microscopies in vivo, can be obtained through the method of the present invention.

To achieve the above object, one aspect of the present invention provides a method for observing, identifying, and detecting blood cells, comprising the following steps: a) providing a system comprising a light source, a first color filter, and a detector; wherein the light source has a central wavelength λ, and the collected signals with the central wavelength shorter than λ can pass through the first color filter; b) radiating the light from the light source on a sample; c) producing third harmonic generation signal with a wavelength of λ/3, second harmonic generation signal with a wavelength of λ/2, and two-photon fluorescence with a wavelength longer than λ/2 from sample under light source illumination; d) directing the above mentioned signal light from the sample to pass through the first color filter; and e) converting the third harmonic generation signal to a corresponding electrical signal by the detector. The system of the present invention can preferably further comprise a first optical splitter, a second optical splitter, and a third optical splitter.

The light source of the present invention is not particularly limited. Preferably, the light source of the present invention is a short pulse laser. More preferably, the light source of the present invention is a femtosecond Cr: forsterite laser. The central wavelength of the light source is not particularly limited, Preferably, the central wavelength of the light source is from 1000 to 1350 nm. More preferably, the central wavelength of the light source is from 1100 to 1300 nm.

In the step c) of the method of the present invention, under light source illumination, sample could also produce second harmonic generation signal with a wavelength of λ/2 and two-photon fluorescence with a wavelength longer than λ/2. In the step d) of the method of the present invention, the light from samples will passes through the optical splitter to separate signals from excitation laser beams. The first color filter will further extinct the excitation light at the wavelength of λ. The signals will then directed to the optical splitter, through which the second harmonic generation light and two-photon fluorescence can be separated after step d) to another detection path. Along this separated detection path, more preferably, the optical splitter is introduced to separate the second harmonic generation signals from the signal having a wavelength longer than λ/2. These two signals are detected separately by different detectors. The type of the color filter applied in the method of the present invention is not limited. Preferably, the color filter is a color glass filter.

In the system of the present invention, the optical splitter is preferred to be a set of dichroic beam splitters.

The system of in step a) of the method of the present invention can optionally further comprise an objective to effectively excite the sample, and collect the signals of second harmonic generation, third harmonic generation, or two-photon fluorescence from the samples.

In step a) of the method of the present invention, the system can optionally further comprises a phase compensator for compensating wave-front distortion caused by surface tissue to improve the optical focusing in the system.

In step a) of the method of the present invention, the system can optionally further comprise a relay lens to avoid the deviation of the laser light beams from the center of lens of the scanner and an entrance center of objective, and reducing difference between the sizes of light beams and the diameter sizes of entrance of objective.

The method of the present invention can preferably optionally further comprise a step f) repeating the steps of b) to e) to process a two-dimensional scanning on the surface of the samples.

The detector applied in the method of the present invention is not limited. Preferably the detector applied in the method of the present invention is a photomultiplier tube. More preferably, three photomultiplier tubes are applied in the step (a) of the system in the method of the present invention.

The method of the, present invention can preferably further comprise a step g) using a microprocessing unit to receive and process the electrical signal, and form and output images of the samples after the step e). The frame rate of the images of the two-dimensional scanning in the method of the present invention is not limited. Preferably, the frame rate of an image of the two-dimensional scanning is more than 30 Hz.

The method of the present invention is preferably used to detect leukocytes and red blood cells. Besides, the method of the present invention is more preferably used to determine types or number of leukocytes per unit volume of blood.

The moving velocity of leukocytes or blood cells can be measured by the method of the present invention. In the method of the present invention, leukocyte amount per unit volume of blood is calculated by the following formula:

n=N/(πR²VT),

wherein R is a radius of a blood vessel;

V is a mean flow velocity of leukocytes;

T is a video time; and

N represents numbers of leukocytes appearing during the video time.

The types of leukocytes can be distinguished by the THG revealed granularity and morphologies according to the method of the present invention. Further, the method of the present invention further can be used to analyze a ratio between cell nucleus and cytoplasm, or to detect the flowing circulation tumors cells in blood.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic view showing a system used in the method of the present invention;

FIG. 2 is a third harmonic generation image of red blood cells under human capillary according to example of the present invention;

FIG. 3 is a third harmonic generation image of round leukocytes under human capillary according to example of the present invention;

FIG. 4 is a third harmonic generation image of neutrophils of the mice according to example of the present invention;

FIG. 5 is a third harmonic generation image of monocytes of the mice according to example of the present invention;

FIG. 6 is a third harmonic generation image of lymphocytes of the mice according to example of the present invention;

FIG. 7 is an intensity distribution of third harmonic generation images of neutrophil, monocyte, and lymphocyte according to the present invention;

FIG. 8 is a time course image combined second harmonic generation (green color shown in figures) and third harmonic generation (magenta color shown in figures) of inflammation microenvironments at 6 hours post lipopolysaccharide challenge; white arrows indicate the leukocytes infiltrating from blood vessel outlined by dashed yellow lines.

FIG. 9 is a time course image combined second harmonic generation (green color shown in figures) and third harmonic generation (magenta color shown in figures) of inflammation microenvironments at 3 days post lipopolysaccharide challenge; white arrows indicate the leukocyte with lymphocyte-like morphology.

FIG. 10 is a time course image combined second harmonic generation (green color shown in figures) and third harmonic generation (magenta color shown in figures) of inflammation microenvironments for at 3 days post lipopolysaccharide challenge; white arrow indicates the leukocyte with lymphocyte-like morphology.

FIG. 11 is a time course image combined second harmonic generation (green color shown in figures) and third harmonic generation (magenta color shown in figures) of inflammation microenvironments at 6 days post lipopolysaccharide challenge; white arrow indicates the blood vessel.

FIG. 12 to FIG. 15 are combined images of second harmonic generation (green color shown in figures) and third harmonic generation (magenta color shown in figures) of subcutaneous microenvironments. SG: sebaceous gland. White arrows in FIG. 13 indicate the region of blood vessels.

FIG. 16 are images of 15 round blood cells captured within 4-minutes in volunteer capillary.

FIG. 17 to FIG. 20 are third harmonic generation images for the analysis of flow velocity of blood cells through a high speed capturing technology.

FIG. 21 are images showing third harmonic generation images of leukocytes from spleen extract and white arrows indicate lymphocyte cells having hollow-core type; (b) is two-photon fluorescence signal, (c) is third harmonic generation signal, and (d) is a combined images of an anti-CD3ε-Allophycocyanin labeled T-lymphocyte; (e) the bright-field image of lymphocyte with Wright-Giemsa stain; (f) the third harmonic generation images of hollow-core leukocytes without anti-CD3ε-Allophycocyanin targeting in spleen extracts.

### DETAILED DESCRIPTION OF THE PREFERRED EMBODIMENT

Hereafter, examples will be provided to illustrate the embodiments of the present invention. Other advantages and effects of the invention will become more apparent from the disclosure of the present invention. Other various aspects also may be practiced or applied in the invention, and various modifications and variations can be made without departing from the spirit of the invention based on various concepts and applications.

### Example 1

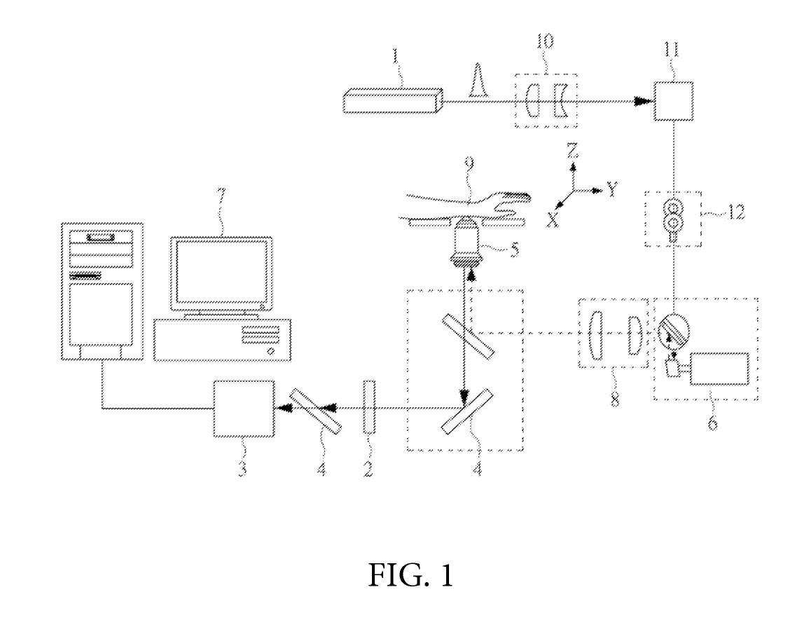

The method described in the present example is a method for retrieving blood cell information by a noninvasive way. With reference to FIG. 1, a schematic view of a system used in the method of the present invention is shown. The method of the present example is executed by following steps. First, a system is provided. The system comprises a light source 1, a first color glass filter 2, and a detector 3. The light source applied in the present example has a central wavelength λ of 1230 nm. A first color glass 2 through which a light having the central wavelength of λ/3 can pass is used in the present example. Then the light from the light source is introduced on a sample 9, and the light from the sample is introduced to pass through an optical splitter 4 to separate into beams. The signal light from the sample 9 is collected and directed to the first color glass filter 2 and a third harmonic generation light having the central wavelength of λ/3 passes. Subsequently, the passed third harmonic generation signal is converted to a corresponding electrical signal by the detector 3. Finally, a microprocessing unit is used for receiving and processing the electrical signal to form or output images of sample to be observed.

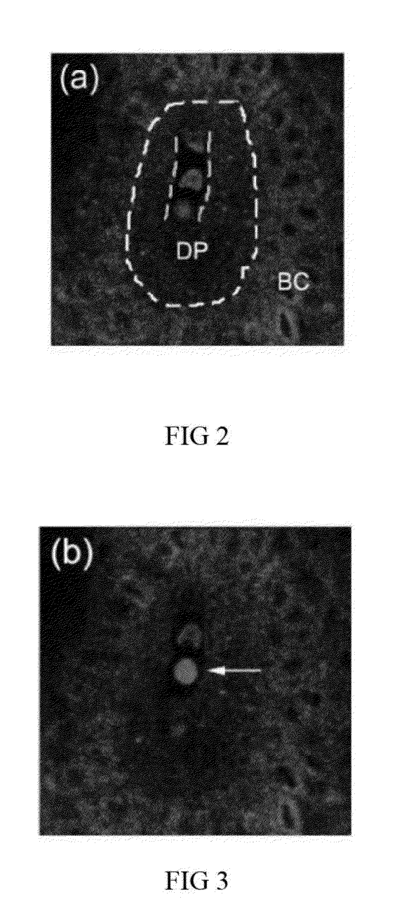

Third harmonic generation microscopy of red blood cells of human capillary in dermal papilla (DP) (outlined by white dashed lines) surrounded by basal cells (BC) (outlined by yellow dashed lines) can be observed by the method described in example 1 and is shown in FIG. 2. The intracellular third harmonic generation can be enhanced by melanin (shown in FIG. 2); therefore, the cytoplasm of basal cells can be revealed clearly. Since the size of human red blood cells are typically 8 μm, within an 85 μm×85 μm field of view, the time it took to scan through them were typically 3 millisecond. For 300 μm/sec circulation speed at deep vessel, blood cells only moved 0.9 μm in each frame, which wouldn't give severe distortion of images. In the course of 30 fps recording (30 images per second), third harmonic generation microscopy constantly captured the images of parachute-shaped red blood cells (RBCs) shown in FIG, 2. The shape of red blood cells, lacking of nuclei, can be predicted by hydrodynamic physics. However, every now and then, round blood cells are observed, which presume they are leukocytes with nuclei and not easy to compress and deform. Therefore, round leukocytes and the parachute-shaped red blood cells in flow can be obviously differentiated by third harmonic generation microscopy. Furthermore, most of the observed round blood cells have much brighter third harmonic generation contrast than RBCs and surrounding basal cells (FIG. 3, pointed by a white arrow). Such bright third harmonic generation contrast could originate from the densely-packed lipid granules inside the white blood cells.

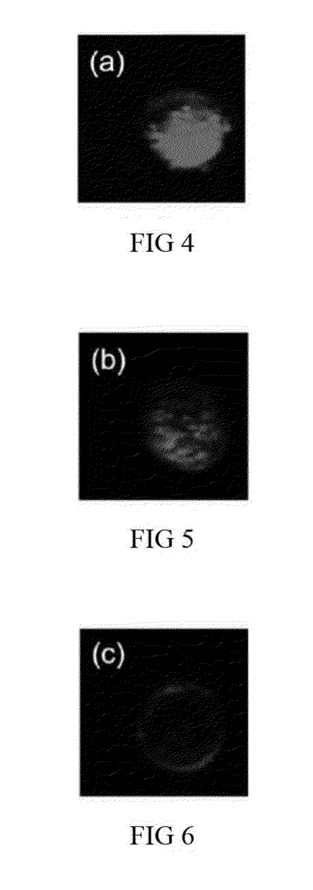

Besides, THG images of mice neutrophils, inonocytes, and lymphocytes (FIG. 4 to FIG. 6) are investigated by the method described in example 1. Neutrophils with high granularity have the most strong THG signals whose granules can be clearly observed. In contrast, lymphocyte showed hollow-core shapes in THG microscopy. Therefore, the type of leukocytes can be further identified by THG contrast based on intensity distribution in cells.

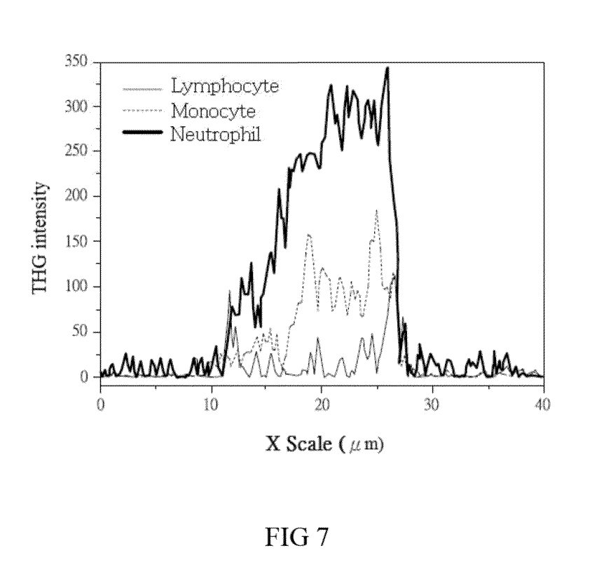

FIG. 7 shows the intensity distribution of third harmonic generation which is observed by third harmonic generation microscopy of example 1. According to FIG. 7, intensity distribution of third harmonic generation in neutrophils, monocytes, and lymphocytes can be observed and analyzed.

A technology that the third harmonic generation contrast is used to identify granularity of leukocytes can be known according to example of the present invention. This is based on the physical mechanism that third harmonic generation nonlinear effects having specific sensitivity to lipid vesicles.

### Example 2

The steps of the method in example 2 are the same as those in example 1, except that the additional detector s used (not shown in figure) after optical splitter 4 to capture second harmonic generation signal. After the lights from samples 9 passing through the optical splitter 4, the second harmonic generation light having the central wavelength of λ/2 are separated into the additional detector 3. Then, the second harmonic generation signal is converted to a corresponding electrical signal by it. Finally, the electrical signal is received and processed by a microprocessing unit to further form output images of second harmonic generation signals of the samples.

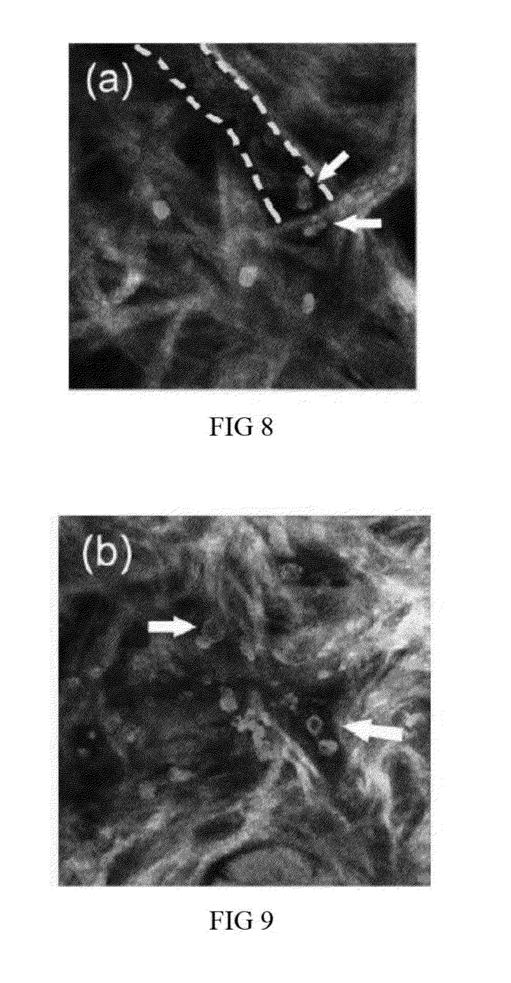

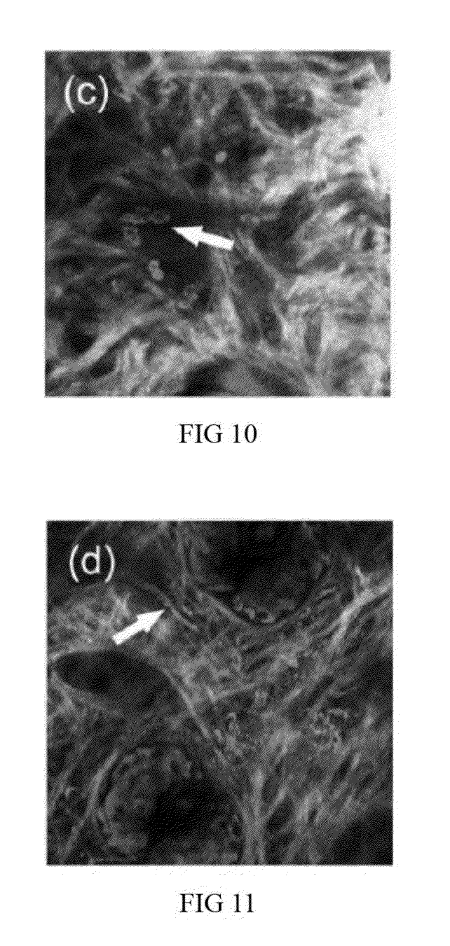

The second harmonic generation images observed by the method described above are shown in FIG. 8 to FIG. 11. FIG. 8 to FIG. 11 are combined second harmonic generation (green color shown in figures) and third harmonic generation (magenta color shown in figures) time course images of inflammation microenvironments of 6 hours, 3 days, and 6 days post-lopopolysaccharide (LPS) challenge. White arrows in FIG. 8 indicate infiltrating and deformed neutrophils, white arrows in FIG. 9 and FIG. 10 indicate hollow-core lymphoid cells, and white arrows in FIG. 11 indicate vessels with circulating red blood cells.

### Example 3

The steps of the method in example 3 are the same as those in example 1, except that the original optical splitter is replaced by a dichroic beam splitter. In addition, an objective 5 is applied and included in the system in the present example. The objective 5 is located under samples for both focusing light and collecting signals. Moreover, three photomultiplier tubes are applied to function as the detector 3 in the present example. One of the photomultiplier tubes is used for detecting the THG signals, one is for second harmonic generation signals, and the other is for detecting the two-photon fluorescence. The second harmonic generation signals, the third harmonic generation signals, and two-photon fluorescence signals from samples 9 are first collected by using the objective 5. The collected signals are directed to the first color glass filter, and a dichroic beam splitter. Then the second harmonic generation signals, the third harmonic generation signals, and two-photon fluorescence signals are separated by dichroic beam splitter, detected by corresponding photomultiplier tubes, and converted to corresponding electrical signals in the present example. Finally, the electrical signal are received and processed by a microprocessing unit to form and output ages of the second harmonic generation signals, the third harmonic generation signals and two-photon fluorescence signals of the samples.

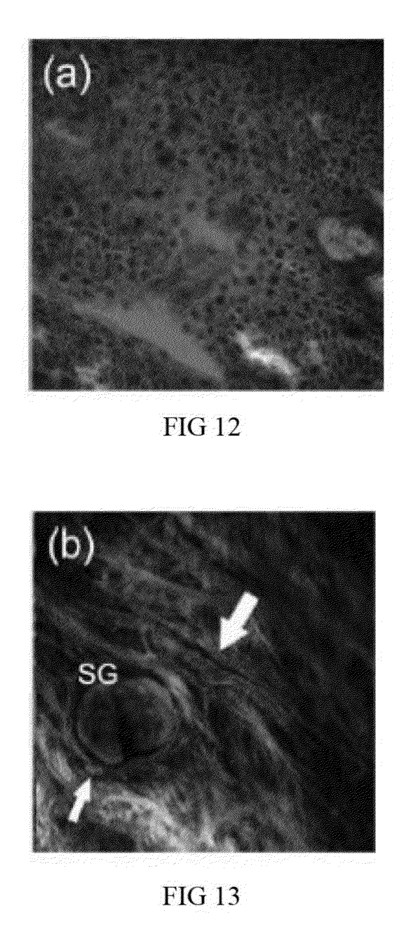

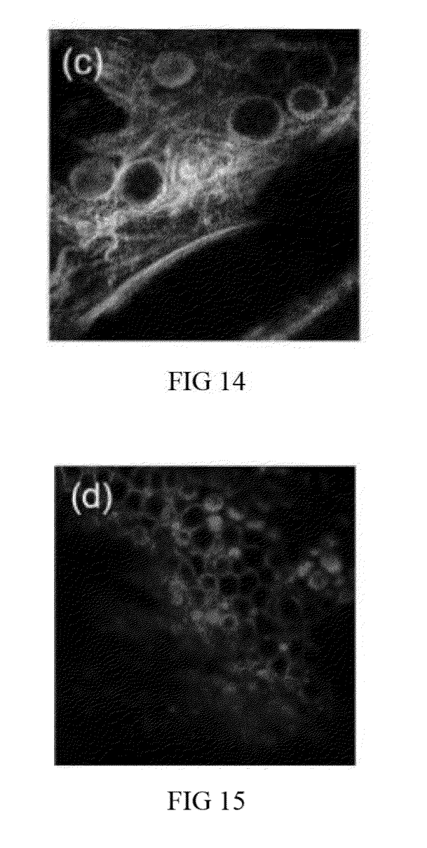

The combined second harmonic generation (green color shown in figures) and THG images (magenta color shown in figures) of subcutaneous microenvironments are observed in FIG. 12 to FIG. 15 according the example 3. FIG. 12 is the images of epithelial keratenocytes, FIG. 13 is the image of vessel network around sebaceous gland (SG). FIG. 14 is the image of adipocytes, and FIG. 15 is the image of chondrocytes. Moreover, the fields of view of FIG. 12 to FIG. 15 are 240×240 μm.

In examples 1 to 3 of the present invention, the light source of the present invention further comprises a telescope 10, made from a concave lens and convex lens. It is used to change the beam spot size and reduce the divergence or convergence angle of light source. Besides, the light source further provides a periscope 11, located in front of an aperture 12, used for changing the height and the polarization of laser light. Then, the light source further provides the aperture 12 which is used for helping alignment of laser beam into scanning unit 6.

In examples 1 to 3, of the present invention, the system further comprises a relay lens 8 made from two lenses, and a set of the relay lens 8 is placed between the scanners 6 and objective 5, so that the scanning pivot and the back aperture of objective will form a pair of conjugated imaging planes. Scanned light beams from scanning pivot will converge to the back aperture center of objective. Furthermore, the beam size will be expanded to fill the size of back aperture.

### Example 4

In the method of the example 4 of the present invention, the steps of the method in example 1 are repeated. The locations of X direction and Y direction on the surface areas of samples are varied after the light beams are focused through the objective to achieve a two-dimension plane scanning and obtain plane-sectioning information, and then two-dimension images are established completely. The example of the present invention provides an frame rate more than 30 Hz; namely, the number of images may reach 30 per second. The example of the present invention may capture images of blood cells at high speed, and may response flowing circulation of blood cells in the blood vessel to measure the velocity of blood flow and cell morphologies. Furthermore, the blood counts per unit volume can be Obtained by the following formula: n=N/(πR²VT); wherein R is the radius of a blood vessel; V is the mean flow velocity; T is a video time; and N represents numbers of leukocytes appearing at the video time. In the calculation, the denominator (πR²VT) represent a total flux of blood in video time T. It can also be calculated by a summation of incremental flux at each frame i by (πR²VᵢΔT), where Vᵢ is the instantaneous velocity of flow and ΔT is the frame period.

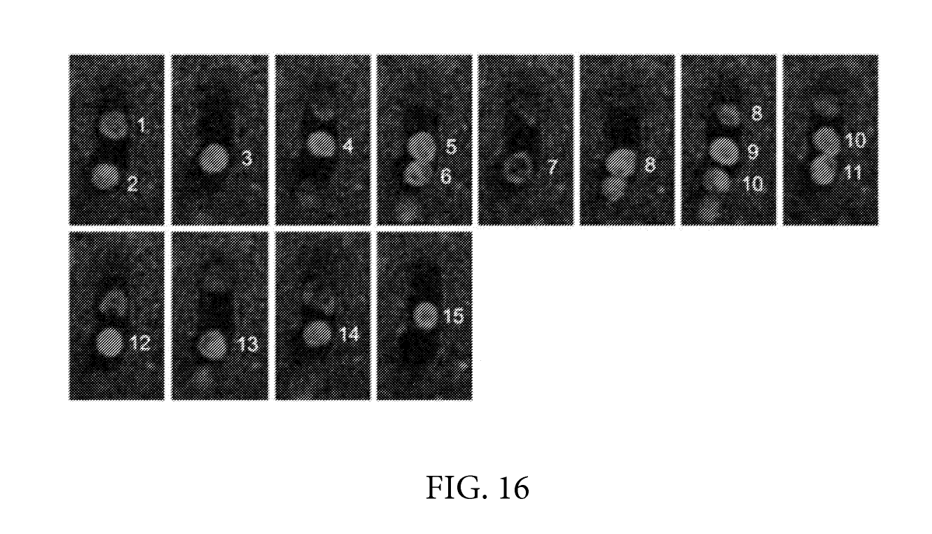

FIG. 16 shows images captured by example 4 of the present invention, within 4-minutes of recording, in the capillary of a volunteer, and 15 round blood cells are captured. The consecutive frames of these images are analyzed to make sure they maintained round shapes in circulation. Cell number 12, also shown in FIG. 3, was the brightest one of third harmonic generation. Other round cells more or less had one or two (number 6 and 8 in FIG. 16) dimed THG regions within cells. Just like the negative contrast in basal cells (such as those in FIGS. 2 and 3), they might be the signatures of nuclei. The number 7 round cell in FIG. 16 could be the lymphocyte with single large nucleus, therefore, a hollow bubble-like third harmonic generation morphologies are revealed.

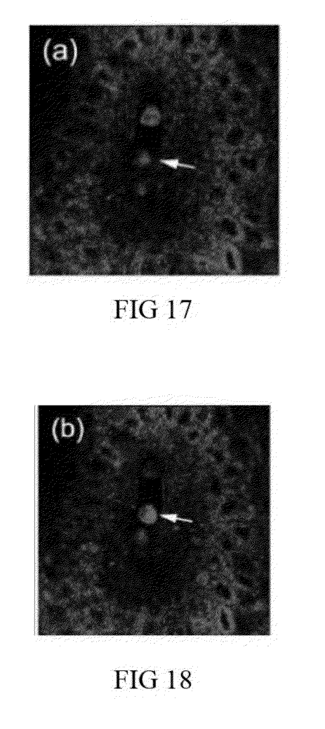

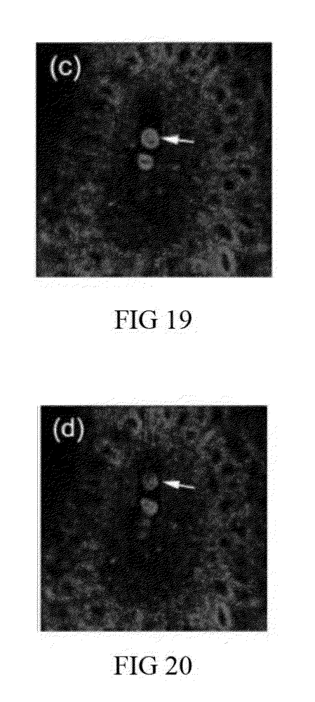

Besides, FIG. 17 to 20 are images captured by example 4 at high speed, and then the velocity of blood flow may be evaluated from the flowing images of one lymphocyte.

### Example 5

In the example 5 of the present invention, all steps of the example 5 are the same as those in example 1, except that the observed samples are labeled or label-free. The samples are treated as following steps: leukocytes from spleen extracts were stained with Allophycocyanin APC-labeled anti-CD3ε antibodies (clone 145-2C11) for 30 min and then washed with 1×PBS buffer (137 mM NaCl, 2.7 mM, KCl, 10 mM Na2HPO4, 2 mM KH2PO4, pH=7.4). Finally, the samples are placed in the system to perform a THG microscopy and a two-photon fluorescence microscopy. The two-photon fluorescence of APC centered at 656 nm falls in the detection window of the third photomultiplier tube, which may confirm that blood cells are T lymphocytes or not. For the convenience of observation with the nonlinear optical microscope, labeled cells were mounted between a cover glass and a slide with 6 μm space in between.

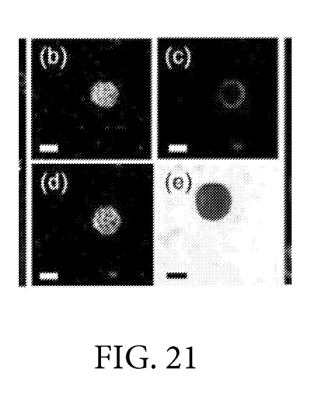

In splenocyte extracts, flow cytometry analysis showed that 50% of leukocytes had the mouse T lymphocyte-specific CD3ε marker. In a typical THG image of splenocyte extract without labeling, 70% of them are found a feature of a hollow core (FIG. 21(a), indicated by white arrows). To confirm the third harmonic generation morphology of T lymphocytes, splenocyte extracts further are immunolabeled with anti-CD3ε-Allophycocyanin (APC), which targets the specific surface CD3ε marker of mouse T lymphocytes.

To avoid interference from strong interface THG, the sectioning images 2˜3 μm away from the water-glass interface are acquired typically. Since cells are close to the surface of glasses, two-photon fluorescence signals excited from the membrane surfaces could still be collected by the third photomultiplier tube, and the average THG intensities in T lymphocytes (FIG. 7, black curve) were one order of magnitude lower than those of neutrophils (FIG. 7, red curve). Compared with the bright-field image of lymphocytes (FIG. 21(e)), this observation might be due to the fact that the nuclei of lymphocytes (stained with magenta color) occupy most of the volume of whole cells. In this labeled extract, some hollow-core cells did not have anti-CD3ε-APC staining [FIG. 21(f)]. These cells might represent other lymphoid cells, such as B lymphocytes or natural killer cells.

These results indicate that leukocytes with different granularities have different morphologies and contrasts in THG microscopy. Neutrophils have extraordinarily high THG contrast that can be easily distinguished from other leukocytes. Lymphoid cells, due to their large single nucleus, have common features of hollow cores and stronger THG contrast at cellular boundaries.

According to the above examples, the method of the present invention may be detected as following: 1) leukocytes can be observed by applying THG contrast; 2) red blood cells and leukocytes are identified by analyzing THG hydrodynamics images; 3) moving velocity of leukocytes is measured by analyzing consecutive THG images; 4) the type of cells are identified by applying intensity distribution of THG contrast in the cells; and 5) leukocyte counts per unit volume of blood is calculated by the following formula: n=N/(πR²VT).

Leukocytes in vivo can be observed by the method of the present invention without labeling, and granularity of leukocytes can be identified. The scope of application may include that evaluating size distribution of red blood cells, identifying local swelling is bacterial-induced or allergic inflammation, and obtaining leukocyte counts per unit volume for tree major types of leukocytes (namely, neutrophils, monocytes, and lymphocytes) without a draw of blood. Because red blood cells may be analyzed by THG images with high resolving capability, the technology may also identify sickle-cell anemia and whether malaria parasites are present in red blood cells or not. Besides, the bloods which have been drawn can be used on present flow cytometry to perform an analysis on volume ratio of cell nucleus over cytoplasm. The scope of application includes that the type of leukocytes are identified without adding antibody and the flowing circulation tumor cells are detected in bloods.

The method of the present invention has effects of optical tomography having a property of minor injury, and the method of the present invention may capture the deepest image depth reaching human skin (>150 μm) while the method of the present invention keeps highest resolution (<500 nm) in vivo by means of microscopy manners.

Although the present invention has been explained in relation to its preferred embodiment, it is to be understood that many other possible modifications and variations can be made without departing from the spirit and scope of the invention as hereinafter claimed.

What is claimed is:

1. A method for observing, identifying, and detecting blood cells, comprising the following steps:
a) providing a system comprising a light source, a first color filter, and a detector; wherein the light source has a central wavelength λ, and the light having a central wavelength shorter than λ can pass through the first color filter;
b) radiating the light from the light source on a sample;
c) under light source illumination, sample could producing third harmonic generation signal with a wavelength of λ/3, second harmonic generation signal with a wavelength of λ/2, and two-photon fluorescence with a wavelength longer than λ/2 from sample under light source illumination;
d) directing the above mentioned signal light from the sample to pass through the first color filter;
e) converting third harmonic generation signal to a corresponding electrical signal by the detector.

2. The method as claimed in claim 1, wherein the central wavelength λ of the light source ranges from 1000 to 1350 nm.

3. The method as claimed in claim 1, wherein the light source is a laser.

4. The method as claimed in claim 1, wherein the light from samples passes through an optical splitter to separate third harmonic generation, second harmonic generation, and two-photon fluorescence after step d).

5. The method as claimed in claim 4, wherein the lights from samples passes through the first color filter and a second harmonic generation light having the central wavelength of λ/2 can be separated by optical splitter.

6. The method as claimed in claim 5, wherein the two-photon fluorescence from samples are furtherseparated by optical splitter after step d).

7. The method as claimed in claim 1, wherein the system in step a) further comprises an objective for focusing the laser lights to excite the samples, and collecting the signals of second harmonic generation, third harmonic generation, or two-photon fluorescence from the samples.

8. The method as claimed in claim 1, further comprising step f) repeating the steps from b) to e) to processing a two-dimensional scanning on the surface of the samples.

9. The method as claimed in claim 8, wherein the frame rate of an image of the two-dimensional scanning is more than 30 Hz.

10. The method as claimed in claim 1, wherein the optical splitter is a set of dichroic beam splitters.

11. The method as claimed in claim 1, wherein the detector of the system is a photomultiplier tube.

12. The method as claimed in claim 1, further comprising step g) using a microprocessing unit to receive and process the electrical signal, and further form and output images of the samples after step e).

13. The method as claimed in claim 1, wherein the method for detecting leukocytes, or red blood cells.

14. The method as claimed in claim 1, wherein the method is used for determining the types or the number of leukocytes per unit volume of blood.

15. The method as claimed in claim 1, wherein the moving velocity of leukocytes can be measured by observing or computing the moving distances between different frames of images of leukocytes.

16. The method as claimed in claim amount per unit volume of leukocytes is calculated by the following formula:
n=N/(πR²VT)
wherein, R is a radius of a blood vessel;
V is a mean flow velocity of leukocytes;
T is a video time; and
N represents numbers of leukocytes appearing during the video time.

17. The method as claimed in claim 1, wherein the types of leukocytes can be distinguished by the THG revealed granularity and morphologies.

18. The method as claimed in claim 1, wherein the method can be used to analyze a ratio of nucleus and cytoplasm.

19. The method as claimed in claim 1, wherein the method can be used for detecting the flowing circulation tumor cells in bloods.
